(19) United States
(12) Patent Application Publication
(10) Pub. No.: US 2022/0162399 A1
(43) Pub. Date: May 26, 2022

(54) MULTIFUNCTIONAL MICROCARRIERS WITH THERMO-RESPONSIVE BIOMATERIAL COATING AND USE THEREOF

(71) Applicant: UVic Industry Partnerships Inc., Victoria (CA)

(72) Inventors: Mohsen Akbari, Victoria (CA); Amir Seyfoori, Tehran (IR); Seyyed Ali Seyyed Ebrahimi, Tehran (IR)

(73) Assignee: UVic Industry Partnerships Inc., Victoria (CA)

(21) Appl. No.: 17/538,714

(22) Filed: Nov. 30, 2021

Related U.S. Application Data
(63) Continuation of application No. 16/007,874, filed on Jun. 13, 2018.
(60) Provisional application No. 62/519,103, filed on Jun. 13, 2017.

(51) Int. Cl. C08J 3/12; A61K 9/51; C12N 13/00; C08J 3/075; C12N 11/10; C12N 11/04; A61K 9/50; C12N 11/087

(52) U.S. Cl. CPC C08J 3/126; A61K 9/5094; A61K 9/5192; C12N 13/00; C08J 3/075; C12N 11/10; C12N 11/04; A61K 9/5026; C12N 11/087; A61K 9/5036; C08J 2433/26; C08J 2389/00; A61K 9/5031; A61K 9/5057; A61K 9/5115

(57) ABSTRACT

A stimulus-responsive carrier, a method for making and a method of using the same are disclosed. The stimulus-responsive carrier comprises a polymeric component comprising poly(N-isopropylacrylamide) (PNIPAM), a copolymer comprising units derived from N-isopropylacrylamide and acrylic acid (PNIPAM-AA), poly N-vinylpyrrolidone, a copolymer of N-isopropylacrylamide and hydroxymethylacrylamide (PNIPAM-HMAAm), a copolymer of N-isopropylacrylamide and allylamine (poly(NIPAAM-co-allylamine)), poly 2-(2-methoxyethoxy) ethyl methacrylate, or any combination thereof; and a second component disposed within the polymeric component, the second component comprising a hydrogel, wherein the second component has a different composition than the polymeric component. The stimulus-responsive carrier is responsive to a stimulus comprising a temperature change, a pH change, application of a magnetic field, or any combination thereof.

### CROSS REFERENCE TO RELATED APPLICATIONS

This application is a continuation of U.S. application Ser. No. 16/007,874, filed Jun. 13, 2018, which claims the benefit of the earlier filing date of U.S. Provisional Patent Application No. 62/519,103, filed on Jun. 13, 2017, each of which is incorporated by reference in its entirety herein.

### FIELD

The present disclosure concerns multi-functional stimulus-responsive carriers, and methods of making and uses thereof.

### SUMMARY

Disclosed herein are embodiments of a stimulus-responsive carrier comprising: a body of a polymeric component comprising poly(N-isopropylacrylamide) (PNIPAM), a copolymer comprising units derived from N-isopropylacrylamide and acrylic acid (PNIPAM-AA), poly N-vinylpyrrolidone, a copolymer of N-isopropylacrylamide and hydroxymethylacrylamide (PNIPAM-HMAAm), a copolymer of N-isopropylacrylamide and allylamine (poly(NIPAAM-co-allylamine)), poly 2-(2-methoxyethoxy) ethyl methacrylate, or any combination thereof; and a second component disposed within the body of the polymeric component, the second component comprising a hydrogel, wherein the second component has a different composition than the polymeric component. The carrier is responsive to a stimulus comprising a temperature change, a pH change, application of a magnetic field, or any combination thereof.

In some embodiments, the polymeric component is covalently bound to a surface of the second component, and the polymeric component comprises at least one tunable property that is selected from an average length of plurality of polymer chains, a surface area density of the plurality of polymer chains on the surface of the second component, an average thickness of the polymer component as measured from the surface of the second component to an outer surface of the polymeric component, a pH-responsive moiety content, or any combination thereof.

In some embodiments, the second component is disposed within the polymeric component to provide a mixture of the second component and the polymeric component. In such embodiments, the polymeric component may comprise at least one tunable property selected from an average length of plurality of polymer chains, a pH-responsive moiety content, or a combination thereof.

In any or all of the above embodiments, the polymeric component may further comprise a surfactant, wherein the surfactant decreases a hydrodynamic diameter of the carrier, increases a surface charge of the carrier, or both.

In any or all of the above embodiments, the second component may comprise a hydrogel selected from a denatured protein, a polysaccharide, a synthetic hydrogel, or any combination thereof. In some embodiments, the hydrogel is methacrylated gelatin polymer (GeIMA), chitosan, collagen type I, collagen type IV, alginate, agarose, hyaluronic acid, elastin, poly(ethylene) glycol (PEG), poly(ethylene glycol) diacrylate (PEGDA), or any combination thereof.

In some embodiments, (i) the polymeric component comprises PNIPAM, and the carrier is a thermo-responsive carrier; (ii) the polymeric component comprises PNIPAM-AA, and the carrier is a thermo-, and pH-responsive carrier; (iii) the polymeric component comprises PNIPAM, the second component further comprises a magnetic nanoparticle, and the carrier is a thermo-, and magnetic-responsive carrier; or (iv) the polymeric component comprises PNIPAM-AA, the second component further comprises a magnetic nanoparticle, and the carrier is a thermo-, pH-, and magnetic-responsive carrier.

In any or all of the above embodiments, the second component may comprise (i) a magnetic nanoparticle, (ii) an active agent, or (iii) both (i) and (ii). In any or all of the above embodiments, the carrier may have (i) an average diameter within a range from 500 nm to 200 μm in a hydrated state, as measured by dynamic light scattering (DLS) technique, (ii) an elastic modulus ranging from 1 kPa to 1 MPa, as measured by atomic-force microscopy, or (iii) both (i) and (ii). In any or all of the above embodiments, the carrier may further comprise a targeting agent bound to the polymeric component.

Also, disclosed herein are embodiments of a method for making a stimulus-responsive carrier comprising a body of a polymeric component comprising poly(N-isopropylacrylamide) (PNIPAM), a copolymer comprising units derived from N-isopropylacrylamide and acrylic acid (PNIPAM-AA), poly N-vinylpyrrolidone, a copolymer of N-isopropylacrylamide and hydroxymethylacrylamide (PNIPAM-HMAAm), a copolymer of N-isopropylacrylamide and allylamine (poly(NIPAAM-co-allylamine)), poly 2-(2-methoxyethoxy) ethyl methacrylate, or any combination thereof; and a second component disposed within the body of the polymeric component, the second component comprising a hydrogel, wherein the second component has a different composition than the polymeric component. In some embodiments, the method comprises: combining a polymeric component with a second component to form the carrier comprising the second component disposed within the body of the polymeric component, wherein the second component has a different chemical composition than the polymeric component.

Also, disclosed herein are embodiments of a method for using a stimulus-responsive carrier comprising a body of a polymeric component comprising poly(N-isopropylacrylamide) (PNIPAM), a copolymer comprising units derived from N-isopropylacrylamide and acrylic acid (PNIPAM-AA), or any combination thereof; and a second component disposed within the body of the polymeric component, the second component comprising a hydrogel, wherein the second component has a different composition than the polymeric component. In some embodiments, the method comprises: administering the carrier to a use environment; and applying a stimulus to the carrier, the stimulus comprising a temperature change, a pH change, application of a magnetic field, or any combination thereof.

The foregoing and other objects, features, and advantages of the invention will become more apparent from the following detailed description, which proceeds with reference to the accompanying figures.

### BRIEF DESCRIPTION OF THE DRAWINGS

FIG. 1 illustrates a schematic representation of synthesis and fabrication of a stimulus-responsive carrier comprising a hydrogel coated with a polymeric component disclosed herein.

FIG. 2 illustrates a schematic representation of in-situ synthesis of a chitosan-magnetic nanoparticle (MNP) hydrogel incorporated within poly (N-isopropylacrylamide)-acrylamide (PNIPAM-AA) polymer matrix disclosed herein.

FIG. 3 is a graphic plot illustrating hydrodynamic diameter variation as a function of temperature for stimulus-responsive carriers comprising (I) PNIPAM-AA, (II) PNIPAM-AA-SDS, and (III) PNIPAM.

FIG. 4 is a graphic plot depicting shrinkage ratio of the stimulus-responsive carriers of FIG. 3 as a function of temperature.

FIG. 5 is a graphic plot depicting hydrodynamic diameter variation as a function of temperature for in-situ synthesized magnetic microcarriers comprising PNIPAM with (e.g., (II) 0.1 wt % of PNIPAM; and (III) 0.3 wt % of PNIPAM, respectively) and without a (I) magnetic hydrogel.

FIG. 6 is a graphic plot depicting shrinkage ratio of the in-situ synthesized magnetic microcarriers of FIG. 5 as a function of temperature.

FIG. 7 is a representative scanning electron microscopic image of in-situ synthesized magnetic microcarriers as disclosed herein.

FIG. 8 is another representative scanning electron microscopic image of in-situ synthesized magnetic microcarriers as disclosed herein.

FIG. 9 is a graphic plot depicting zeta potential profile as a function of pH for (1) chitosan-coated magnetic nanoparticles and (II) a magnetic stimulus-responsive carrier as disclosed herein.

FIG. 10 is two photographs showing the physical phase transition between sol to gel state above LSCT of a PNIPAM-AA solution at pH=7.

FIG. 11 is a vibrating sample magnetometer plot of magnetic-chitosan nanogel as disclosed herein showing magnetization as a function of magnetic field.

FIG. 12 is a vibrating sample magnetometer plot showing magnetization as a function of magnetic field for multi-functional pH-magneto microcarriers developed with different methods as disclosed herein.

### DETAILED DESCRIPTION

### I. Definitions

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 1997 (ISBN 0-471-29205-2).

In order to facilitate review of the various embodiments of the disclosure, the following explanations of specific terms are provided:

Carrier: As used herein in its simplest meaning, a carrier is a particle comprising a body of a polymeric component and a second component disposed within the body of the polymeric component.

Coil-to-globule transition: As used herein, “coil-to-globule transition” refers to collapse of a polymer from an expanded coil state through an ideal coil state to a collapsed globule state, or vice versa.

Copolymer: A polymer formed from polymerization of two or more different monomers.

Denatured protein: A protein in which chemical composition and/or stereochemical structure are altered such that the biological activity of the denatured protein is substantially minimized or absent from the biological activity of the original protein. In some embodiments, the chemical composition and the stereochemical structure of the denatured protein are altered by external physical means such as, heat, radiation, or any combination thereof. In some embodiments, the chemical composition and the stereochemical structure of the denatured protein are altered by chemical reaction, such as reaction with strong acids, alcohols, or any combination thereof. Exemplary denatured proteins may include, but are not limited to, methacrylated gelatin (also may be referred to herein as “GeIMA”), methacrylated collagen (also may be referred to herein as “Col-MA”), or any combination thereof.

Dispersity: A measure of the heterogeneity of particle sizes in a population of particles. Particles are considered to be monodisperse if the particles have roughly the same size, shape, and/or mass, e.g., a deviation in size or mass of less than 10% relative to the average size or mass. Particles are considered to be polydisperse if the size, shape, and/or mass distribution is variable.

Gel: A colloidal system comprising a solid three-dimensional network within a liquid. By weight, a gel is primarily liquid, but behaves like a solid due to a three-dimensional network of entangled and/or crosslinked molecules of a solid within the liquid. From a rheological perspective, a gel has a storage modulus G′ value which exceeds that of the loss modulus G″. The storage modulus is a measure of the energy stored in a material in which a deformation (e.g., sinusoidal oscillatory shear) has been imposed; storage modulus can be thought of as the proportion of total rigidity of a material that is attributable to elastic deformation. The loss modulus is a measure of the energy dissipated in a material in which a deformation (e.g., sinusoidal oscillatory shear) has been imposed; loss modulus can be thought of as the proportion of the total rigidity of a material that is attributable to viscous flow rather than elastic deformation. The storage modulus and loss modulus can be determined with a rheometer.

Hydrogel: A cross-linked three-dimensional network of polymeric chains that are capable of absorbing and retaining molecules (e.g., water, polar solvents, non-polar solvents, drugs in liquid form, or the like) in their three-dimensional networks. Hydrogel-forming polymeric chains comprise one or more hydrophilic functional groups in their polymeric structures, such as amino (NH₂), hydroxyl (OH), amide (—CONH—, —CONH₂), sulfate (—SO₃H), or any combination thereof, and can be natural-, or synthetic-polymeric-based networks. In some embodiments, the polymeric chains can comprise a plurality of the same monomeric units. In other embodiments, the polymeric chains can comprise a plurality of different monomeric units. Exemplary hydrogels may include, but are not limited to, proteins (e.g., collagen, gelatin, or the like), denatured proteins (e.g., methacrylated gelatin [GeIMA], methacrylated collagen [Col-MA], or the like), polysaccharide (chitosan, starch, alginate, or the like), synthetic hydrogels (e.g., poly(ethylene glycol) diacrylate [PEGDA]).

Lower Critical Solution Temperature: A critical temperature below or at which a hydrogel can undergo a change from its hydrophilic state to its hydrophobic state, or vice versa. In some embodiments, hydrogel is hydrated below its LCST, and therefore is hydrophilic. In some embodiments, hydrogel is at least partially dehydrated above its LCST, and therefore is insoluble and hydrophobic. Lower Critical Solution Temperature is also referred to herein as “LCST”. In some embodiments, LCST of linear thermo-responsive polymers is determined using cloud point (CP), and is generally used for physically crosslinked polymers. As used herein, “cloud point” refers to temperature at the outset of cloudiness, the temperature at inflection point of a transmittance curve, or the temperature at a defined transmittance. The cloud point can be affected by many structural parameters of the hydrogel like the hydrophobic content, architecture of the hydrogel, molar mass of the hydrogel, or any combinations thereof.

Magnetic nanoparticle: A nanoparticle that can be manipulated using magnetic fields.

Multiparticulate composition: A composition comprising a plurality of discrete particles.

pH-Responsive polymer: A polymer that responds to changes in the pH of the surrounding medium by varying its conformation. In some embodiments, the polymer undergoes a change its conformation from an elongated coil to a more collapsed globule.

Polymer: A molecule of repeating structural units (e.g., monomers) formed via a chemical reaction, i.e., polymerization. In some embodiments, polymers are synthetic polymers, such as poly(ethylene glycol) (also may be referred to herein as “PEG”), poly(N-isopropylacrylamide) (also may be referred to herein as “PNIPAM”), poly(ethylene glycol) diacrylate (also may be referred to herein as “PEGDA”) or any combination thereof. In some embodiments, polymers are naturally derived polymers, such as gelatin, collagen, alginate, heparin, carob gum, or any combination thereof.

Polysaccharide: A polymeric carbohydrate molecules comprising long chains of monosaccharide units (e.g., 10 or more units) bonded together by glycosidic linkages, which on hydrolysis provide the constituent monosaccharides and/or oligosaccharides. Exemplary polysaccharides include, but are not limited, chitosan, chitin, cellulose, glycogen, or the like.

Stimulus-responsive polymer: A polymer that undergoes a change in its structure, e.g., from a hydrophilic state to a hydrophobic state, triggered by an external stimulus that alters the environment of the stimulus-responsive polymer. External stimuli can include, but are not limited to, heat, pH, ionic strength, magnetic field, electrical field, light, ultrasound, chemical species, or any combination thereof. In particular embodiments, external stimuli include heat, pH, ionic strength, magnetic field, or any combination thereof. In some embodiments, the change in the polymer is a reversible change. In some embodiments, the change in the polymer is an irreversible change.

Target: An intended molecule to which a carrier comprising a targeting agent is capable of specifically binding. Examples of targets include cell-surface proteins and other antigens, such as proteins and other antigens on the surface of tumor cells.

Targeting agent: An agent capable of binding to a specific binding partner, i.e., a target. Exemplary targeting agents include antibodies, antibody fragments, affibodies, aptamers, albumin, cytokines, lymphokines, growth factors, hormones, enzymes, immune modulators, receptor proteins, antisense oligonucleotides, avidin, nano particles, and the like. Particularly useful of targeting agents are antibodies, although any pair of specific binding partners can be readily employed for this purpose.

Thermo-Responsive polymer: A polymer that exhibits a volume phase transition at a certain temperature, which results in an abrupt change in the solubility of the polymer.

Volume-phase transition temperature (VPTT): A critical temperature below or at which a hydrogel undergoes a change from swelling to shrinking. In some embodiments, the hydrogel can swell below the critical temperature and collapse above the critical temperature. Volume-phase transition temperature is also referred to herein as “VPTT”, and is typically used with reference to chemically crosslinked polymers, such as hydrogels. VPTT of thermo-responsive polymers may be determined using the equilibrium swelling ratio method, e.g., as disclosed by Varghese et al. (Sensors and Actuators B: Chemical 2008, 135:336-341) and Zhang et al. (Acta Biomaterialia 2009, 5:488-497).

Zeta-potential: A potential difference existing between a surface of a solid particle immersed in a conducting liquid and the bulk of the liquid.

### II. Stimulus-Responsive Carrier

Disclosed herein are embodiments of a stimulus-responsive carrier as well as methods of making and using the same. In some embodiments, the stimulus-responsive carrier is a multi-functional stimulus-responsive carrier, i.e., the carrier responds to two or more stimuli. The stimulus-responsive carrier disclosed herein is a particle comprising two or more components and having one or more physical and/or chemical properties that can be tuned to provide the carrier with responsiveness (or degree of responsiveness) to one or more external stimuli. In some embodiments, the tunable physical or chemical property may abruptly change in response to small changes in one or more external stimulus, such as a change in temperature, a change in pH, or any combination thereof. In certain embodiments, the tunable physical or chemical property may facilitate manipulation of the carrier, e.g., movement of the carrier in response to application of a magnetic field. In some embodiments, property changes are reversible. That is, the stimulus-responsive carrier can revert back to its original state once the external stimulus is removed. In certain embodiments, property changes are irreversible (e.g., release of an active agent within the carrier). In still other embodiments, one or more properties may undergo a reversible change and another property undergoes an irreversible change. Advantageously, the tunable physical and/or chemical properties of the stimulus-responsive carrier disclosed herein allow the carrier to be suitable for use with a variety of applications, such as cell culturing systems, drug-delivery systems, antibody production, applications involving capturing or isolating cell-related components, such as cells, protein, exosome, mRNA, etc., in biological samples, as implantable materials that are suitable for use in biomedical implants, and the like.

The stimulus-responsive carrier comprises a body of a polymeric component. By “body” is meant a volume of the polymeric component and does not include, for example, individual polymers dispersed in a solution.

In certain disclosed embodiments, the stimulus-responsive carrier comprises a body of a polymeric component comprising a thermo-responsive polymer that can undergo a coil-to-globule transition and/or a phase separation at its lower critical solution temperature (LCST) in aqueous solution. As such, the resulting stimulus-responsive carrier can be responsive to a change in external stimulus, such as a change in temperature. That is, the thermo-responsive polymer of the stimulus-responsive carrier can impart a reversible transition from swollen hydrated state to a shruken dehydrated state at or above its LCST, while the thermo-responsive polymer of the stimulus-responsive carrier can hydrate to its swollen state below its LCST. Exemplary thermo-responsive polymers of the stimulus-responsive carrier may include, but are not limited to, poly (N-isopropyl acrylamide) (PNIPAM), a copolymer of N-isopropylacrylamide and acrylic acid (PNIPAM-AA), poly N-vinylpyrrolidone, a copolymer of N-isopropylacrylamide and hydroxymethylacrylamide (PNIPAM-HMAAm), a copolymer of N-isopropylacrylamide and allylamine (poly(NIPAAM-co-allylamine)), poly 2-(2-methoxyethoxy) ethyl methacrylate, or the like. The polymer can have any suitable molecular weight, depending on the intended type of application of the stimulus-responsive carrier. In some embodiments, the polymer of the stimulus-responsive carrier has an average molecular weight from 50,000 to 70,000 Daltons, such as 60,000 to 70,000 Daltons, or 60,000 to 65,000 Daltons.

In certain embodiments, the stimulus-responsive carrier comprises a polymeric component comprising a copolymer of a thermo-responsive monomer and a pH-responsive monomer. The resulting stimulus-responsive carrier can undergo a phase separation with change in pH at specific temperatures (e.g., at a temperature from 25° C. to 37° C.), as well as a phase separation with a change in temperature. For example, an increase in pH can lead to a significant increase in LCST of the stimulus-responsive carrier, presumably due at least in part to the ionization of the —COOH groups present in the pH-responsive monomer. Increase in temperature can, in turn, impart a reversible transition from a swollen hydrated state to a shrunken dehydrated state to the stimulus-responsive carrier, thereby rendering the resulting carrier to be thermo- and/or pH-responsive carrier. In one example, a thermo-responsive monomer may include, but is not limited to, N-isopropylacrylamide, N-vinylpyrrolidone, 2-(2-methoxyethoxy)ethyl methacrylate, or the like, while a pH-responsive monomer may include, but is not limited to, acrylic acid, allylamine, hydroxymethyl acrylamide, or the like. In particular disclosed embodiment, the stimulus-responsive carrier can be a copolymer of poly (N-isopropyl acrylamide) (PNIPAM) and acrylic acid (AA).

Additionally, or alternatively, in some embodiments, the pH-responsiveness and/or thermo-responsiveness of the stimulus-responsive carrier can also be tuned, for example, by modulating a ratio of the thermo-responsive monomer to that of the pH-responsive monomer present in the stimulus-responsive carrier. For example, a molar ratio of thermo-responsive monomer to pH-responsive monomer present in stimulus-responsive carrier may be within a range from 99:1 to 90:10. In some embodiments, the copolymer of the stimulus-responsive carrier has an average molecular weight from 60,000 to 73,000 Daltons.

In some embodiments, the polymeric component comprises, consists essentially of, or consists of PNIPAM, PNIPAM-AA, poly N-vinylpyrrolidone, a copolymer of N-isopropylacrylamide and hydroxymethylacrylamide (PNIPAM-HMAAm), a copolymer of N-isopropylacrylamide and allylamine (poly(NIPAAM-co-allylamine)), poly 2-(2-methoxyethoxy) ethyl methacrylate, or any combination thereof. By “consists essentially of” means that the polymeric component does not include any other polymer and does not include any other component that may alter the stimulus-responsive properties of the polymeric component. Thus, for example, when the polymeric component consists essentially of PNIPAM and/or PNIPAM-AA, trace amounts of salts or water may be present, but other components such as surfactants, pH modifiers, magnetic compounds, and the like are absent or present in amounts of less than 1 wt % based on the mass of the polymeric component.

The stimulus-responsive carrier comprises second component disposed within the body of the polymeric component. The second component comprises a hydrogel. In one embodiment, a single volume or body of the second component is disposed within the body of the polymeric component such that the second component is partially or entirely embedded within the polymeric component, a core-shell configuration. In an independent embodiment, the second component is disposed with the body of the polymeric component as a plurality of discrete second component bodies or domains. In another independent embodiment, the second component is disposed throughout the body of the polymeric component, thereby forming a mixture of second component molecules and polymeric component molecules.

In some embodiments, the hydrogel component comprises a cross-linked three-dimensional polymeric network of polymers comprising hydrophilic groups that are capable of absorbing and/or retaining molecules (e.g., water, polar solvents, non-polar solvents, drugs in their liquid form, or the like) in their native functional state within the hydrogel scaffold. Suitable hydrogels include, but are not limited to, methacrylated gelatin polymer (GeIMA), chitosan, collagen type I, collagen type IV, alginate, agarose, hyaluronic acid, elastin, poly(ethylene) glycol (PEG), poly(ethylene glycol) diacrylate (PEGDA), or any combination thereof. The-linked three-dimensional network, advantageously, renders tunable a surface stiffness of the stimulus-responsive carrier. Surface stiffness may be expressed in terms of elastic modulus, e.g., Young's modulus. In some embodiments, the carrier has a surface having an elastic modulus ranging from 1 kPa to 1 MPa as measured by atomic force microscopy. Advantageously, the surface stiffness can be tuned to approximate the stiffness of a cellular environment, e.g., brain, connective tissue, muscle, bone, etc. When the stimulus-responsive carrier is used, for example, in cell culture, surface stiffness of the stimulus-responsive carrier can significantly affect cellular viability and proliferation rate of anchorage-dependent cells, such as fibroblasts or stem cells. If the surface is too stiff or insufficiently stiff, cells may adhere poorly to the surface, may proliferate poorly, and/or may even die.

The surface stiffness of the carrier may depend, in part, on the chemical composition of the hydrogel, the molecules retained within the hydrogel and/or a lack thereof, or a combination thereof. Thus, the hydrogel may be selected to provide a carrier with a low, medium, or high surface stiffness. For example, gelatin or methacrylated gelatin (GeIMA) may be used to provide a carrier with a low surface stiffness, poly(ethylene glycol) diacrylate (PEGDA) may be used to provide a carrier with a high surface stiffness, and chitosan may be used to provide a carrier with a medium surface stiffness. In one embodiment, the hydrogel is a methacrylated gelatin having Young's modulus within a range from 5 to 30 kPa, depending on the degree of methacrylation of gelatin. In another embodiment, the hydrogel is chitosan having Young's modulus within a range from 20 kPa to 200 kPa. In yet another embodiment, the hydrogel is poly(ethylene glycol) diacrylate (PEGDA) having Young's modulus within a range from 100 kPa to 1,000 kPa. It is understood that hydrogels other than GeIMA, chitosan, and PEGDA also may be used to provide the carrier with a desired surface stiffness.

Further, the composition of the hydrogel can be modulated so as to define tunable mechanical stiffness of the hydrogel component which, in turn, can provide tunable surface stiffness of the stimulus-responsive carrier. That is, a hydrogel unit having low mechanical stiffness can be combined with a hydrogel unit having moderate mechanical stiffness or a hydrogel having high mechanical stiffness to provide a hydrogel with a tunable mechanical stiffness. In another example, a hydrogel unit having moderate mechanical stiffness can be combined with a hydrogel unit having high mechanical stiffness. Thus, in some embodiments, the hydrogel of the stimulus-responsive carrier is a mixture of two or more hydrogel polymers (e.g., GelMA/chitosan, chitosan/PEGDA, and the like), thereby allowing further variations in the surface stiffness of the carrier. The tunable mechanical stiffness of the hydrogel component provides the stimulus-responsive carrier with a tunable elastic modulus within a range from 1 kPa to 1 MPa, such as, 5 kPa to 50 kPa, or 100 kPa to 300 kPa.

Additionally, or alternatively, any free amino groups or carboxylic groups, if present, of the hydrogel can also facilitate the stimulus-responsive carrier to respond to both acidic and basic pH conditions, thereby rendering the carrier pH-responsive. In some embodiments, the hydrogel of the stimulus-responsive carrier is any hydrogel derived from a naturally-occuring biomaterial, such as protein (e.g., a denatured protein, such as methacrylated gelatin [GeIMA], methacrylated collagen [Col-MA], collagen type I, collagen type IV, elastin, or any combination thereof), a glycosaminoglycan (e.g., hyaluronic acid), a polysaccharide (e.g., chitosan, alginate, agarose, cellulose, or the like), a synthetic hydrogel (e.g., poly(vinyl alcohol), polyacrylamide, poly (ethylene oxide), poly (ethylene glycol) [PEG], poly(ethylene glycol) diacrylate [PEGDA], or the like), or any combination thereof.

In one embodiment, the polymeric component is combined with a hydrogel component, such that the resulting carrier is a mixture of both polymeric component molecules and the hydrogel component molecules. The mixture may be a homogeneous or heterogeneous mixture. In one embodiment, the mixture is a molecular dispersion of the polymeric component molecules and hydrogel component molecules. In such embodiments, surface stiffness of the stimulus-responsive carrier can be tuned, for example, by modulating a ratio of the polymeric component to that of the hydrogel component.

In some embodiments, the stimulus-responsive polymer comprises a body of a polymeric component, and one or more hydrogel bodies or particles dispersed within the body of the the polymeric component such that the hydrogel body or bodies are partially or fully embedded within the polymeric component. The hydrogel bodies may be dispersed randomly and/or uniformly within the body of the polymeric component. In some embodiments, hydrogels dispersed within polymeric component are monodisperse hydrogel particles having roughly same size, shape and/or mass distributions. In some embodiments, hydrogel bodies dispersed within the body of the polymeric component are polydisperse hydrogel particles having variable size, variable shape, and/or variable mass distributions.

When the second component comprises one or more hydrogel bodies dispersed within the body of the polymeric component, the polymeric chains of the polymeric component may be adsorbed or bound (ionically or covalently) to a surface of the hydrogel body or bodies. In some embodiments, the hydrogel component constitutes a core (or a plurality of cores), and the polymeric chains of the polymeric component are disposed thereover constituting a shell of the stimulus-responsive carrier. In such embodiments, the polymeric chains have one or more tunable properties, such as an average length of the polymeric chains, a surface area density of the polymer chains on the surface of the second component, an average thickness of the polymeric component as measured from the surface of the second component to an outer surface of the polymeric component, a pH-responsive moiety content (e.g., an acrylic acid monomer content), or any combination thereof. Advantageously, the polymeric component comprises, consists essentially of, or consists of a thermo-responsive polymer as previously described. The polymeric component also may be pH-responsive. A person of ordinary skill in the art will understand that the hydrogel component of the stimulus-responsive carrier, either as a core or as dispersed components within the polymeric component, also can render the stimulus-responsive carrier to undergo a phase separation in response to a change in external stimulus, as described further below.

Advantageously, the one or more components (e.g., polymeric component, or hydrogel) can also facilitate discretely tuning one or more physical and/or chemical properties in response to a small change in external stimulus, such as a change in temperature, a change in pH, or any combination thereof, thereby defining a multi-functional responsive property of the stimulus-responsive carrier. For example, and as described above, in the case of a stimulus-responsive carrier comprising a thermo-responsive polymer and a hydrogel, the stimulus-responsive carrier can undergo a conformational change (e.g., a phase separation of the polymeric chains and molecules, such as water, within the polymeric network) at its lower critical solution temperature (LCST) in aqueous solution. As such, thermo-responsive polymer of the stimulus-responsive carrier can impart a reversible transition from swollen hydrated state to a shruken dehydrated state at or above its LCST, while the thermo-responsive polymer of the stimulus-responsive carrier can hydrate to its swollen state below its LCST. Thus, the resulting stimulus-responsive carrier comprising thermo-responsive polymer and the hydrogel can be responsive to a change in temperature with a resulting change in a hydrodynamic diameter of the carrier. Additionally, the surface stiffness can be tuned by selection of the hydrogel composition as described above.

In another example, and as described above, in the case of stimulus-responsive carrier comprising (i) a polymeric component comprising a copolymer of a thermo-responsive monomer and a pH-responsive monomer, and (ii) a second component comprising a hydrogel, the resulting stimulus-responsive carrier can undergo a conformational change with change in pH at specific temperatures (e.g., at a temperature from 25° C. to 37° C.), rendering the carrier both temperature and pH responsive. For example, any free —NH₂ group and/or —CO₂H groups, if present, in the polymeric component and/or the hydrogel of the stimulus-responsive carrier can respond to both acidic and basic conditions. In one example, an increase in pH can lead to a significant increase in LCST of the stimulus-responsive carrier, e.g., due to the ionization of the —COOH groups present in the pH-responsive monomer. The increase in LCST can, in turn, impart a reversible transition from swollen hydrated state to shrunken dehydrated state of the stimulus-responsive carrier. Similarly, a decrease in pH can lead to a significant decrease in LCST of the stimulus-responsive carrier, e.g., due to the presence of —NH₂ groups present in hydrogel. The decrease in LCST can, in turn, impart a reversible transition from shrunken dehydrated state to swollen hydrated state of stimulus-responsive carrier. Thus, the stimulus-responsive carrier comprising (i) a pH-responsive hydrogel, and/or (ii) a copolymer of a thermo-responsive monomer and a pH-responsive monomer and a hydrogel can be responsive to a change in temperature, a change in pH, or any combination thereof. Additionally, the surface stiffness can be tuned by selection of the hydrogel composition as described above.

In some embodiments, the polymeric component and/or the hydrogel further comprises a surfactant. Inclusion of a surfactant may decrease the hydrodynamic diameter and/or increase the surface charge of the stimulus-responsive carrier. Increasing the surface charge may increase the LSCT of the polymeric component. Suitable surfactants include nonionic and ionic surfactants. Exemplary surfactants include, but are not limited to sodium dodecyl sulfate, PEGylated fluorosurfactants, cetyltrimethylammonium bromide (CTAB), and sorbitan esters (e.g., Span® 20 sorbitan laurate, Span® 80 sorbitan oleate, Tween® 20 polyethylene glycol sorbitan monolaurate, Tween® polyoxyethylene sorbitan monooleate, and the like).

The stimulus-responsive carrier can have any size that is suitable for its intended use, e.g., for intercellular and/or intracellular interactions. In some embodiments, the stimulus-responsive carrier has an average hydrodynamic diameter within a range from 500 nm to 500 μm in its hydrated state, such as from 500 nm to 200 μm. When the stimulus-responsive carrier undergoes a transition from the swollen, hydrated state to the shrunken, dehydrated state, the diameter may decrease by up to 50%, such as a decrease of from 10-50%, 20-50%, or 30-50%. Thus, a hydrated stimulus-responsive carrier having a diameter of 100 μm may shrink to a diameter of from 50-90 μm in response to a temperature and/or pH change. In one embodiment, the stimulus-responsive carrier is a microcarrier with an average diameter within a range from 50 μm to 500 μm. In another embodiment, the stimulus-responsive carrier is a nanocarrier with an average diameter within a range from 400 nm to 700 nm.

In some embodiments, the second component of the stimulus-responsive carrier further comprises one or more magnetic nanoparticles disposed within the body of the polymeric component or within the hydrogel of the second component. In some embodiments, the magnetic nanoparticle is coated with the hydrogel to form a hydrogel-coated magnetic nanoparticle. In some embodiments, a plurality of the hydrogel-coated magnetic nanoparticles is randomly dispersed within the body of the polymeric component of the stimulus-responsive carrier. In an independent embodiment, a plurality of magnetic nanoparticles is coated with hydrogel to form a single hydrogel body comprising a plurality of magnetic nanoparticles within the hydrogel body. The single hydrogel body is disposed within the body of the polymeric component. In another independent embodiment, a plurality of hydrogel bodies, each hydrogel body comprising a plurality of magnetic nanoparticles within the hydrogel body, is dispersed within the body of the polymeric component.

In some embodiments, the magnetic nanoparticles or hydrogel-coated magnetic nanoparticles are monodisperse particles with substantially the same size, shape, and mass distribution, e.g., varying by less than 10% relative to an average size or mass. In other embodiments, the magnetic nanoparticles or hydrogel-coated magnetic nanoparticles are polydisperse particles with variable size, shape, and/or mass distribution. In another embodiment, the hydrogel-coated magnetic nanoparticle(s) constitute a core of the carrier, and the polymeric component is bound covalently to a surface of the hydrogel-coated magnetic nanoparticle. For example, PNIPAM or PNIPAM-AA can be covalently bound to chitosan-coated magnetic nanoparticles or grafted on the chitosan-coated magnetic nanoparticles, e.g., through atom-transfer radical polymerization.

In another embodiment, each of the magnetic nanoparticle(s) and the hydrogel body or bodies are discretely dispersed within the body of the polymeric component. In yet another embodiment, each of the magnetic nanoparticle(s), hydrogel body or bodies, and the polymeric molecules of the polymeric component are together in a mixture to form the stimulus-responsive carrier.

Exemplary magnetic nanoparticles include magnetic particles of any composition, such as particles comprising elemental iron, nickel, cobalt, or the like, or compounds comprising magnetic elements. In some examples, the magnetic nanoparticles of the stimulus-responsive carrier comprise iron, such as magnetite (Fe₃O₄). The hydrogel for use with the magnetic nanoparticles can be any of the hydrogels described above, the polymeric component can comprise any thermo-responsive polymer or copolymer comprising a thermo-responsive monomer and a pH-responsive monomer as described above, and the magnetic nanoparticles can be any of the magnetic nanoparticles as defined above. In certain embodiments, the hydrogel is a polysaccharide-hydrogel, such as chitosan-hydrogel, and the magnetic nanoparticles are magnetite; the magnetic nanoparticles may be coated with the chitosan. The chitosan-coated magnetic nanoparticle can further be coated with a thermo- and/or pH-responsive polymer or copolymer, such as PNIPAM. PNIPAM-AA copolymer, poly N-vinylpyrrolidone, a copolymer of N-isopropylacrylamide and hydroxymethylacrylamide (PNIPAM-HMAAm), a copolymer of N-isopropylacrylamide and allylamine (poly(NIPAAM-co-allylamine)), poly 2-(2-methoxyethoxy) ethyl methacrylate, or any combination thereof. In some embodiments, the chitosan-coated magnetic nanoparticle is further coated with PNIPAM or PNIPAM-AA.

When the stimulus-responsive carrier comprises magnetic nanoparticles, the stimulus-responsive carrier can undergo a conformational change with a change in external stimulus, such as a change in pH, a change in temperature, or both. Additionally, the carrier can be manipulated by application of an external magnetic field. A magnetic field can be used, for example, to attract and collect one or more stimulus-responsive carriers dispersed in a use environment. Additionally, the surface stiffness of the particle can be tuned by selection of the hydrogel composition.

In some embodiments, the stimulus-responsive carrier further comprises an active agent disposed within the body of the polymeric component or within the hydrogel. Exemplary active agents include, but are not limited to, drugs, growth factors, cytokines, aptamers, peptides, dye molecules, or the like. In some embodiments, the active agent may diffuse out of the carrier over a period of time. In certain embodiments, application of a stimulus to the stimulus-responsive carrier releases at least a portion of the active agent from the carrier. For example, a temperature and/or pH change triggering a conformational change in the polymeric component or the hydrogel may release at least a portion of the active agent from within the carrier. In an independent embodiment, application of an oscillating magnetic field may disrupt the carrier structure, thereby releasing at least a portion of the active agent.

In some embodiments, the stimulus-responsive carrier further comprises a targeting agent or plurality of targeting agents on an outer surface of the carrier. Exemplary targeting agents include, but are not limited to, antibodies, antibody fragments, aptamers, peptides, or the like, that are capable of binding to a target, e.g., an antigen, a receptor, etc. For example, the targeting agent may be an antibody capable of binding to an antigen or ligand on a cell surface. Such embodiments are useful, for example, for binding to particular cells in a medium comprising a plurality of different cells, such as a blood sample comprising red blood cells, white blood cells, and tumor cells. If the stimulus-responsive carrier further comprises magnetic nanoparticles, application of an external magnetic field may be used to isolate the stimulus-responsive carriers and cells bound thereto.

### IV. Methods for Making Stimulus-Responsive Carrier

Disclosed herein are embodiments of methods for making stimulus-responsive carrier. The disclosed methods allow for synthesis of a stimulus-responsive carrier comprising one or more components with one or more physical and/or chemical properties that can be tuned to respond to small changes in one or more external stimuli, such as a change in temperature, a change in pH, application of a magnetic field, or any combination thereof. In some embodiments, a stimulus-responsive carrier comprises (i) a body of a polymeric component comprising a thermo-responsive polymer, a copolymer of a thermo-responsive monomer and a pH-responsive monomer, or a combination thereof, and (ii) a second component comprising a hydrogel. The stimulus-responsive carrier may further comprise magnetic nanoparticles, an active agent, a targeting agent, or any combination thereof. Exemplary polymeric components, hydrogels, magnetic nanoparticles, active agents, and targeting agents are described above. In certain embodiments, the hydrogel component comprises methacrylated gelatin (GeIMA), poly(ethylene glycol) (PEG), poly(ethylene glycol) diacrylate (PEGDA), chitosan, GeIMA/PEG, GeIMA/PEGDA, GeIMA/chitosan, PEG/chitosan, or PEGDA/chitosan, while the polymeric component comprises PNIPAM, PNIPAM-AA, or a combination thereof.

In some embodiments, the polymeric component and hydrogel of the stimulus-responsive carrier are synthesized via conventional methods known to one of ordinary skill in the art of polymer chemistry, such as radical polymerization, photopolymerization, enzymatic reactions, covalent cross-linking, or any combination thereof. In other embodiments, polymeric components and hydrogels disclosed herein are commercially-available.

In some embodiments, one or more primary amino groups of a naturally-occurring protein, such as gelatin, can be converted to an acryloyl derivative by reacting with a corresponding α, β-unsaturated carbonyl compound. The acryloyl derivative can be subsequently subjected to one or more cross-linking reactions to form a hydrogel comprising a denatured protein via a free radical photopolymerization. In one example, radical photopolymerization can be accomplished in the presence of a photoinitiator, a surfactant, UV irradiation, or any combination thereof. In certain embodiments, the cross-linking reactions may optionally be performed in the presence of a synthetic hydrogel, such as poly(ethylene glycol) diacrylate (PEGDA), to obtain a hydrogel comprising a complex of denatured protein and synthetic hydrogel. In certain other embodiments, the cross-linking reactions may optionally be performed in the presence of a polysaccharide, such as chitosan, to obtain a hydrogel comprising a complex of denatured protein and polysaccharide. In some embodiments, primary amino groups of gelatin are at least partially methylated, e.g., by reaction with methacrylate anhydride, to form a corresponding methacryloyl substituted-gelatin, such as GeIMA. A person of ordinary skill in the art will understand that degree of methacrylation can be modulated by varying the number of moles of methacrylate anhydride that react with gelatin. In one example, the methacryloyl substituted-gelatin monomer can have a degree of methacrylation within a range from 20% to 80%, such as 20% to 40%, or 40% to 80%. GeIMA can be subsequently crosslinked to form a hydrogel comprising three-dimensional network of polymeric chains.

In some embodiments, a polymeric component are grafted to a surface of a hydrogel component using techniques such as atom-transfer radical polymerization (ATRP) or surface initiation plasma treatment. In particular disclosed embodiments, the ATRP synthesis of the stimulus-responsive carrier comprising the polymeric component and the hydrogel, is catalyzed using one or more metal complexes in the presence of nitrogen-containing initiators. Advantageously, the grafting of the polymeric component on the surface of the hydrogel component allows the polymeric chains to have one or more tunable properties, such as an average length of the polymeric chains, a surface area density of the polymer chains on the surface of the second component, an average thickness of the polymeric component as measured from the surface of the second component to an outer surface of the polymeric component, a pH-responsive moiety content (e.g., an acrylic acid monomer content), or any combination thereof.

In another embodiment, a polymeric component is adsorbed onto a surface of a hydrogel body by, e.g., dispersing the hydrogel body in a solution of PNIPAM and/or PNIPAM-AA polymeric chains and then collecting the polymer-coated hydrogel body. The polymeric coponent may be crosslinked after adsorption to the hydrogel body. In yet another embodiment, polymeric molecules of the polymeric component and the hydrogel are simply mixed together.

In some embodiments, a hydrogel component comprising a polysaccharide is reacted with the polymeric component under conditions effective to covalently bind the polymeric component to the hydrogel comprising polysaccharide. In one example, covalent binding of the polymeric component can be accomplished in the presence of a carbodiimide, a cross-linker, a surfactant, an additive, or any combination thereof. Exemplary carbodiimides include, but are not limited to, 1-ethyl-3-(3-dimethylaminopropyl)carbodiimide (EDC), N,N′-dicyclohexylcarbodiimide (DCC), or any combination thereof. Exemplary additives may include, but are not limited to, N-hydroxy succinimide (NHS), N-hydroxybenzotriazole, or any combination thereof. Exemplary cross-linkers may include, but are not limited to, glutaraldehyde, formaldehyde, tri-polyphosphate, or any combination thereof. In such embodiments, surfactants, if present in the resultant carrier, can decrease a hydrodynamic diameter of the stimulus-responsive carrier, increase a surface charge of the stimulus-responsive carrier, or any combination thereof.

In some embodiments, a hydrogel component comprising a polysaccharide can be reacted with a magnetic nanoparticle under reaction conditions effective to form a hydrogel-coated magnetic nanoparticle. For instance, chitosan may be combined with magnetic nanoparticles in an acidic (e.g., acetic acid) solution in the presence of a surfactant (e.g., CTAB) under conditions effective to coat the nanoparticles with the chitosan, followed by crosslinking the chitosan (e.g., with glutaraldehyde) to provide hydrogel-coated magnetic nanoparticles. The hydrogel-coated magnetic nanoparticles can subsequently be combined with the polymeric component in the presence of a base or an acid, and under reaction conditions that are effective to coat the hydrogel-coated magnetic nanoparticle with the polymeric component to form a stimulus-responsive carrier. In one embodiment, the polymeric component (e.g., PNIPAM or PNIPAM-AA) is adsorbed to the hydrogel-coated magnetic nanoparticles by electrostatic adsorption, and covalent bonding is subsequently performed, e.g., using carbodiimide chemistry. In some embodiments, the crosslinking reactions can be accomplished in the presence of a surfactant and a cross-linker. Each of the exemplary surfactants and cross-linkers can be any of the surfactants and cross-linkers, respectively, as defined above.

An active agent may be incorporated into the stimulus-responsive carrier. The active agent may be incorporated into the hydrogel body and/or into the body of the polymeric component. In one embodiment, an emulsion of the active agent and the hydrogel polymer chains is prepared to form a hydrogel body comprising the active agent. The hydrogel polymer chains are optionally crosslinked. In another embodiment, an emulsion of the active agent and the polymeric component polymer molecules is formed and used to coat the hydrogel body. The polymeric component polymer molecules may be optionally crosslinked and/or covalently bound to the hydrogel body.

A targeting agent may be incorporated into the stimulus-responsive carrier. In some embodiments, the targeting agent is bound to an outer surface of the stimulus-responsive carrier, such as to an outer surface of the polymeric component. Suitable targeting agents include, for example, antibodies, antibody fragments, aptamers, peptides, nucleotides, and the like. The targeting agent may be bound to the outer surface by conventional methods known to those skilled in the art.

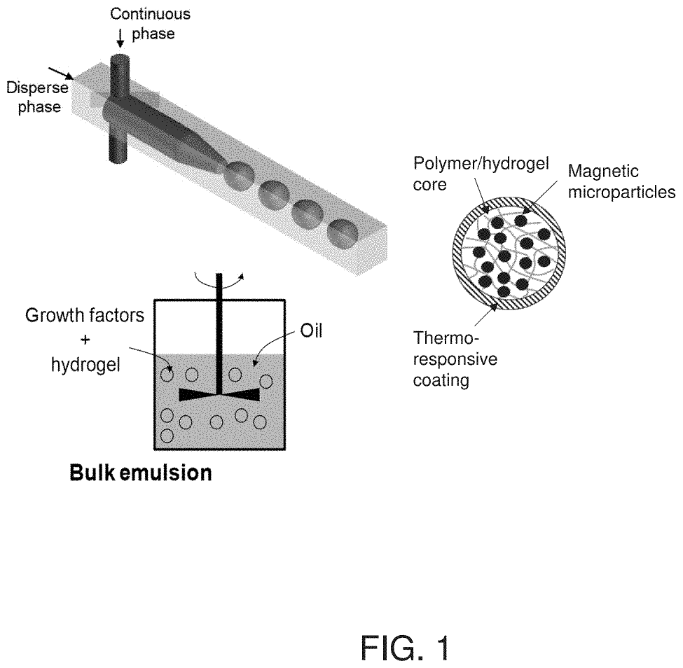

FIG. 1 illustrates one exemplary embodiment in which a stimulus-responsive carrier comprising an active agent is synthesized. An emulsion of active agent and hydrogel is formed such that the active agent (e.g., a growth factor) is dispersed within a body of the hydrogel (e.g., GeIMA/PEG). The polymers of the hydrogel may be crosslinked. The hydrogel body comprising the hydrogel and the active agent is disposed within a body of the polymeric component to form the stimulus-responsive carrier.

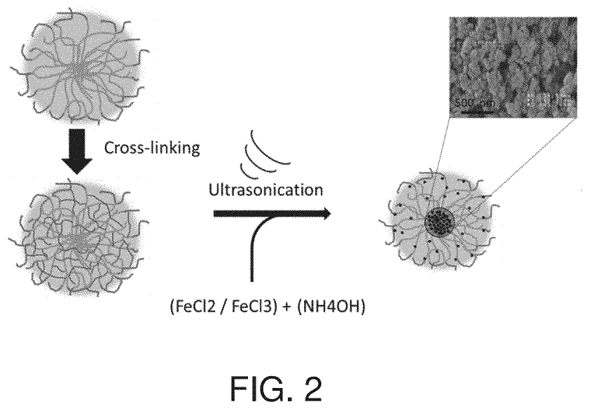

FIG. 2 illustrates another exemplary embodiment in which magnetic nanoparticles are formed in situ by combining magnetic nanoparticle precursors with a solution comprising polymers of the polymeric component and/or the hydrogel component followed by reduction to precipitate magnetic nanoparticles within the polymeric component and/or hydrogel component. The polymers of the polymeric component and/or hydrogel component may be crosslinked prior to or after formation of the magnetic nanoparticles.

### V. Uses of Stimulus-Responsive Carriers

Advantageously, the stimulus-responsive carrier disclosed herein can be tuned to achieve a desired surface stiffness and a desired responsiveness to a change in external stimulus, such as a change in temperature, a change in pH, application of a magnetic field, or any combination thereof. Suitable uses for the disclosed stimulus-responsive carriers include, but are not limited to, cell culturing systems, antibody production, cell/protein/exosome/mRNA capturing in biological samples for diagnostic applications, implantable materials useful for biomedical implants, and wound management.

Embodiments of a method for using the disclosed stimulus-responsive carriers include administering the carrier to a use enviroment, and applying a stimulus to the carrier, wherein the stimulus comprises a temperature change, a pH change, application of a magnetic field, or any combination thereof. In one embodiment, the second component of the carrier comprises a hdyrogel, the stimulus comprises a temperature change, a pH change, or any combination thereof, and applying the stimulus changes a diameter of the carrier. In another embodiment, the second component further comprises an active agent, and applying the stimulus further releases at least a portion of the active agent from the carrier.

In some embodiments, the use environment is a cell culture medium comprising cells, and the method further comprises incubating the cell culture medium at an effective temperature for an effective period of time whereby the cells proliferate and at least some of the cells adhere to the polymeric component of the carrier, the hydrogel, or both the polymeric component and the hydrogel of the carrier, and subsequently applying the stimulus changes a diameter of the carrier thereby releasing at least some of the adhered cells from the carrier. The tunable surface properties of the hydrogel can facilitate adhering and proliferating cells in the cell culture medium, while tunable physical and/or chemical properties of polymeric component facilitate changing a diameter of the carrier, thereby releasing at least some of the adhered cells from the stimulus-responsive carrier. Advantageously, the tunable surface properties allow a surface stiffness of the stimulus-responsive carrier to approximate different cell environments. For example, bone stem cells reside in a stiff environment, while the brain cells reside in a soft environment. As such, surface stiffness of the stimulus-responsive carrier can be tailored to the stiffness of the native environment by modulating, for example, the composition of the hydrogel.

Additionally, active agents such as drugs, growth factors, cytokines, etc. may be incorporated into the carrier. The active agent may be released over time via diffusion from the stimulus-responsive carrier, or the active agent may be released in response to an external stimulus, such as a temperature change, a pH change, an oscillating magnetic field (when magnetic nanoparticles are also included within the carrier), or a combination thereof. In some embodiments, the active agent may be delivered to cells over extended periods, such as a period of hours, days, or even weeks. Such ability allows delivering active agents at the interface of cells and the substrate at desired concentration, significantly reducing the amount of costly active agents in large-scale cell cultures. Advantageously, the stimulus-responsive carriers greatly increase an available surface growth surface per volume of the cell culture medium as compared to traditional cell culture flasks, which can significantly enhance harvest densities of the cells without increasing volumetric footprint of the cell culture apparatus. In some embodiments, an enzyme-free cell harvesting approach using a stimulus-responsive carrier as disclosed herein can reduce cell death rate and can result in high quality cells without damaged surface proteins.

In one embodiment, the stimulus-responsive carrier is used for growing cells from a pure population in cell culture, as the cells can bind to a denatured protein hydrogel, such as GeIMA), without binding, if present, to either the polysaccharide hydrogel or the synthetic hydrogel. In one embodiment, the carrier comprises a mixture of GeIMA polymers and PNIPAM/PNIPAM-AA polymers rather than having a core-shell configuration. In another embodiment, the carrier has a core-shell configuration and further comprises a plurality of targeting agents on the outer surface of the carrier. For example, the carrier may comprise a plurality of antibodies, the antibodies capable of binding to the cells in the cell culture.

In any or all of the foregoing embodiments, the second component may further comprise a magnetic nanoparticle, and applying the stimulus may comprise applying a magnetic field, wherein applying the magnetic field induces a movement of the carrier. Such embodiments facilitate isolation of the carrier from the use environment. For example, application of the magnetic field may attract the carriers and adhered cells from a cell-culture medium, thereby facilitating manipulation, isolation, and/or concentration of the carriers with the attached cells. Subsequent application of a temperature and/or pH change can be used to trigger a change in the diameter of the carrier, thereby releasing at least some of the adhered cells.

In certain embodiments, the use environment is a biological sample comprising a target cell, the carrier further comprises magnetic nanoparticles and a targeting agent capable of binding to the target cell, and the method further comprises waiting an effective period of time prior to applying the stimulus to allow binding of the targeting agent to the target cell, thereby forming a carrier-cell complex, and applying a magnetic field induces movement of the carrier-cell complex, whereby the carrier-cell complex is isolated from the biological sample. In one embodiment, such carriers are used in a diagnostic method to detect presence of a target cell within the biological sample, such as detecting circulating tumor cells in a blood sample. In another embodiment, the carrier can be used to isolate target cells from the biological sample, whereby the carrier-cell complex can be introduced to a cell culture medium to induce proliferation of the target cells.

In one embodiment, the stimulus-responsive carrier is used for wound management. The carrier may include a pH-responsive hydrogel and/or a pH-responsive polymeric component. Advantageously, the carrier may further comprise an active agent, such as an antibacterial agent or a pain-relieving agent. The carrier may be applied to the wound. In response to a pH change and/or a temperature change, the carrier may release at least a portion of the active agent into the wound. In some instances, bacteria in a wound may alter the pH of the wound environment, thereby releasing an antibacterial agent from the carrier only if the wound is infected.

In another embodiment, the stimulus-responsive carrier is pH-responsive and further comprises an active agent, such as a drug, and the use environment is the gastrointestinal tract. As the carrier transits from the low-pH gastric environment to the higher pH intestinal environment, the pH change triggers a conformational change in the carrier, thereby releasing the active agent from the carrier into a desired portion of the gastrointestinal tract.

In still another embodiment, the stimulus-responsive carrier is temperature-responsive and further comprises an active agent. The carrier may be incorporated into a biomedical implant. After implantation in a subject, the resulting temperature change triggers a conformational change in the carrier, thereby releasing the active agent from the carrier.

### VI. Examples

### Example 1

### Synthesis of Methacrylated Gelatin Hydrogel

Gelatin was mixed at 10% (w/v) with Dulbecco's phosphate-buffered saline (DPBS; Gibco) at 50° C. and stirred until completely dissolved. Methacrylation of gelatin was achieved by adding 20% (w/v) of methacrylic anhydride (MA) to the reaction mixture using conventional synthetic methods. In one example, methacrylic anhydride (MA) was added at a rate of 0.5 mL/min under stirred conditions at 50° C. and allowed to react for 2 hours. After diluting with 5×DPBS to stop the reaction, the mixture was dialyzed against distilled water using 12-14 kDa cutoff dialysis tubing for 1 week at 40° C. to remove salts and unreacted methacrylic acid. The solution was lyophilized for 1 week to generate a white porous foam and was stored at −80° C.

Table 1 depicts several examples of complete varying materials and parameters utilized in the synthesis of hydrogels:

TABLE 1
varying materials and parameters utilized in the synthesis of hydrogels

| | | Degree of | Photo- | Weight ratio |
|---|---|---|---|---|
| | Matrix | meth- | initiator | Matrix: |
| Carriers | (w/v) % | acrylation | (w/v) % | 2^{nd} phase |
| GelMA (high | 5%, 10%, | 80%, 20% | 0.25%, | 1 |
| methacrylation) | 15% | | 0.5%, 0.8% | |
| GelMA (low | 5%, 10%, | 80%, 20% | 0.25%, | 1 |
| methacrylation) | 15% | | 0.5%, 0.8% | |
| PEGDA | 5%, 10% | — | | 1 |
| GelMA/PEGDA | 10%, 30%, | 20% | 0.5% | 1 |
| | 60% | | | |
| PNIPAM- | 5%, 10%, | 20% | 0.5% | 1:0.2, 1:0.4, 1:0.6 |
| AA@GelMA | 15% | | | |
| PNIPAM-coated | 5%, 10%, | 20% | 0.5% | |
| GelMA | 15% | | | |

### Example 2

### Synthesis of Thermo- and pH Responsive PNIPAM-AA Polymeric Component

1.13 g N-isopropyl acrylamide (NIPAM) (10 mmol), 50 mg N, N-methylene bisacrylamide (BIS) (0.32 mmol), and acrylic acid (0.3 mM) were dissolved in 90 mL filtered, de-ionized water in a three-necked flask that has been equipped with a magnet stirrer and purged with nitrogen for 20 minutes. 60 mL of this solution was then filled in a syringe. 10 mL water was added to the remaining 30 mL solution in the flask, and the liquid was heated to 80° C. and purged with nitrogen. The precipitation polymerization was initiated by addition of 27 mg ammonium persulfate (APS) (0.05 m mol) dissolved in 2 mL of water. After about 4 minutes, when the solution started to become turbid, the solution in the syringe was fed into the reaction vessel at a rate of 1 mL/min. After 4 hours, the nitrogen purging was stopped and the whole medium was purified to avoid collection of unpolymerized monomers and other contaminants using dialysis for about 1 week in de-ionized water.

### Example 3

### Fabrication Routes of GeIMA/PEGDA Carriers with PNIPAM Shell Using Droplet Micro-Fluid Method

Droplet microfluidic devices made from polydimethyl siloxane (PDMS) using soft lithography are employed to fabricate monodisperse solid polymeric particles and GeIMA carriers. This is accomplished by forming monodisperse pre-hydrogel drops and polymerizing the monomers or crosslinking the polymers within these drops. To ensure monodisperse drop formation, a flow focusing geometry is patterned into these devices. The mechanisms by which these drops gel are categorized as chemical gelation, temperature-change induced gelation, coalescence-induced gelation, and ionic gelation using internal and external crosslinking. Polymerization of monomers and/or gelation of polymers in these methods is initiated by ultraviolet (UV) irradiation, heat transfer, or chemical transport within and out of the hydrogel droplets.

Table 2 shows materials and parameters of a droplet microfluid method utilized in fabricating stimulus-responsive carrier

TABLE 2
Varying Droplet microfluidic methods parameters

| | Continuous | Dispersed | | |
|---|---|---|---|---|
| Carriers | phase | phase | Surfactant | Photoinitiator |
| GelMA (high | Mineral oil | Polymer + | 20% wt | Irgacure ® |
| methacrylation) | | magnetic | Span 80 | 2959 |
| & (low | | microgel | | |
| methacrylation) | | | | |
| PEGDA | HFE-7500 oil | Polymer + | PEGylated | Irgacure ® |
| | | magnetic | fluoro- | 2959 |
| | | microgel | surfactant | |
| GelMA/PEGDA | Mineral oil | Polymer + | Span 80 | Irgacure ® |
| | | magnetic | | 2959 |
| | | microgel | | |

### Example 4

### Atom-Transfer Radical Polymerization to Synthesize Stimulus-Responsive Carrier with GeIMA and PEGDA Core and PNIPAM Shell

Polymer chains are grafted to the GeIMA surface through graft to (atom transfer radical polymerization, ATRP) route. In this method, the ATRP mechanism is catalyzed by Cu(I)/Cu(II) complexes in which Azo-initiators start the radical polymerization on the surface of the substrate matrix as well as maintaining the amount of Cu ion for propagating of the polymerization. In this regard, PNIPAM polymer chains are grafted to the surface with tunable length, thickness and densities. A mixture of Si—Br (0.5 g), CuCl (21 mg), tris(2-dimethylaminoethyl)amine (48 mg), and N-isopropyl acrylamide (NIPAm) (2.36 g) was added to the solvents of dimethyl formamide/water (DMF/H₂O) (1:1 v/v, 5 ml). The mixture was purged with nitrogen for 30 min. The polymerization was performed for 6 hours at the room temperature in the presence of nitrogen. The final product was obtained after being filtrated, washed with water, methanol, acetone, and dried at reduced pressure at 35° C.

### Example 5

### Synthesis of Multi-Functional Magnetic-Nanoparticle (MNP)-Chitosan Comprising poly(N-isopropylacrylamide)-acrylic acid (PNIPAM-AA) Carriers

The multi-functional magnetic-nanoparticle (MNP)-chitosan complex comprising poly(N-isopropylacrylamide)-acrylic acid (PNIPAM-AA) carrier can be assembled using a two-step synthetic process described below.

Step 1 comprises coating Fe₃O₄ nanoparticles with chitosan hydrogel to form a magnetic-nanoparticle (MNP)-chitosan complex, while step 2 comprises infiltration of magnetic-nanoparticle (MNP)-chitosan complex within a matrix of poly (N-isopropylacrylamide)-acrylic acid (PNIPAM-AA) microgel to covalently bind PNIPAM-AA copolymer matrix to magnetic (MNP)-chitosan complex. The hybrid-nanoparticle complex microcarrier thus synthesized can act as pH-magneto sensitive materials with the ability to respond to both acidic and basic pH conditions due to the presence of both amino (NH₂) group of the chitosan and carboxylic acid group of PNIPAM-AA copolymer.

### a) Fabrication of Magnetic-pH-Responsive Hydrogel

For synthesizing pH-responsive carriers containing magnetic nanoparticles, 5% wt chitosan was dissolved in 1% v/v acetic acid solution. To a pre-synthesized Fe₃O₄ magnetic nanoparticle suspension, 20 mg/mL cetrimonium bromide (CTAB) surfactant was added. This solution was added dropwise to the vigorously stirred (800 rpm) solution of chitosan and sonicated for 1 hours. Then 3 ml of glutaraldehyde was added, and after about 20 minutes the solution was washed with distilled water and absolute ethanol (water:ethanol 50:50) and finally dried in a vacuum oven at 100° C. for 6 hours.

### b) Synthesis of Thermo-pH-Magnetic Responsive Carriers Comprising Magnetic-Nanoparticle (MNP)-Chitosan Comprising poly(N-isopropylacrylamide)-acrylic acid (PNIPAM-AA) Copolymer

Thermo-pH-magnetic-responsive carriers can be synthesized using two methods as disclosed below:

### i) Physical Absorption and Chemical Bonding Method

To form a multifunctional (thermo-pH-magnetic) microcarrier, the synthesized magnetic-nanoparticle (MNP)-chitosan complex was added dropwise to a specific concentration of the poly (N-isopropylacrylamide)-acrylic acid (PNIPAM-AA) copolymer solution at about 2:1 wt % ratio and sonicated for another 2 hours. Infiltration of the MNP-chitosan complex in the PNIPAM-AA copolymer matrix was conducted using electrostatic absorption of the positively-charged chitosan of the chitosan-coated MNP and negatively charged PNIPAM-AA copolymer matrix at pH of about 5. After mixing for 1 hour by ultrasonication in ice bath, chitosan-coated MNP are located in the negative charge zones of the PNIPAM-AA copolymer matrix and hence the net charge is decreased. Then, covalent bonding is performed using carbodiimide chemistry by adding 0.5 mg 1-ethyl-3-(3-dimethylaminopropyl)carbodiimide (EDC) and 0.5 mg N-hydroxy succinimide (NHS) respectively. The mixture is sonicated in the ice bath for another 1 hour to ensure a reaction of the amino groups of the chitosan with carboxylic groups of the PNIPAM-AA copolymer matrix. The final product is purified by magnetic decantation and further washing and centrifuging with DI water for 3 times.

### ii) In-Situ Synthesis Method

In-situ Fe₃O₄ nanoparticles were synthesized using FeCl₂.4H₂O and FeCl₃.6H₂O (molar ratio 1:2) within 3D matrix of poly(N-isopropylacrylamide)-acrylic acid (PNIPAM-AA) copolymer microgel. To acquire different amounts of magnetic nanoparticle loading within the matrix of the PNIPAM-AA copolymer microgels, 2 different microgel suspension with concentration of 0.1 wt % and 0.3 wt % were prepared in DI water at pH=6 and subsequently, ferrite precursors containing iron (II) and Iron (III) chloride hexahydrate with molar ratio of 1/2 were added to 50 ml of the microgel suspension and mixed using mechanical stirring under N₂ atmosphere for 2 hours. After about 2 hours, ammonium hydroxide (NH₄OH) was added dropwise to the above mixture followed by increasing the stirring rate from 400 to 1000 rpm. The nanoparticle sedimentation reaction was completed in an hour and consequently, the magnetic microgels were decanted magnetically and purified using dialysis for 2 days by every day media changing.

### Characterization of the Stimulus-Responsive Carriers:

Depending on the type of the carrier, different methods were used to analyze the physical and chemical characteristics of the fabricated carriers, as described below.

Hydrogel components, namely, GeIMA and PEGDA, of the stimulus-responsive carrier are analyzed using Scanning electron microscopy (SEM) and Dynamic light scattering methods, while thermo and pH-responsive components of the stimulus-responsive carriers are investigated through Zeta potential and DLS analysis over different temperatures or pH conditions. Further, the rate of magnetic responsiveness, if present, of these microgels are measured using vibrating sample magnetometer (VSM). The results of the various analytic methods are described below. Still further, chemical analysis of the stimulus-responsive carrier is conducted through spectral analysis, such as FTIR, NMR.

Additionally, or alternatively, stiffness and elastic modulus of the GeIMA/PEGDA hydrogel component of the stimulus-responsive carrier has been conducted by force measurements using atomic force microscopy (AFM)-assisted nanoindentation. The experimental setup consists of the AFM placed on top of an inverted optical microscope by which monitoring of the AFM cantilever and the microgel sample during indentation measurement is allowed. The cantilever was initially positioned at the center of the microgel, and then lowered at certain rate of 3 to 5 μm s⁻¹ to indent the carrier. The applied force (F) is measured as a function of the position of the cantilever. The elastic modulus (E) is calculated using formula provided below based on Hertz contact mechanics theory for the spherical elastic solid:

F=π(E/1−v²)R1/2h3/2

where R is radius of the carrier sphere, h is the indentation depth, and v is the Poisson's ratio.

Further, Table 3 shows materials and the whole process of the microcarrier synthesis. Due to having homogenous crosslinking density through entire volume of the microcarrier in contrast to usual one-batch synthesis method, semi-batch method was applied in which a specific amount of the monomer, cross-linker and surfactant were added drop-wise to the certain amount of primary solution of these precursors a few minutes after polymerization starting. In the semi-batch method in contrast to one-batch method, cross-linker and surfactant are distributed homogenously in the entire structure of the forming microgel and hence the mechanical properties distribution of the whole microgel are more homogenous.

TABLE 3
Materials and process parameters for synthesis of stimulus-responsive
carriers:

| | | | | | Hydro- |
|---|---|---|---|---|---|
| | | BIS | | | dynamic |
| | Monomer/ | (cross- | | Zeta | size |
| | AA | linker) | SDS | potential | (dₕ) |
| Microcarriers | (Molar ratio) | (mmol) | (mmol) | (mV) | (nm) |
| PNIPAM | 10/0 | 0.2 | 0 | −3.6 | 530 |
| PNIPAM-AA (1) | 10/0.5 | 0.2 | 0 | −8.54 | 568 |
| PNIPAM-AA (2) | 10/0.5 | 0.2 | 0.12 | −16.2 | 510 |
| MNP-Chitosan | — | — | — | 18.4 | 84 |
| MNP- | 10/0.5 | 0.2 | 0.2 | −7.40 | 628 |
| Chitosan@PNIPAM- | | | | | |
| AA | | | | | |
| In-situ magnetic | 10/0.5 | 0.2 | 0.2 | −22 | 670 |
| microgel | | | | | |

Hydrodynamic diameter of the carriers in Table 3 was measured using dynamic light scattering (DLS) method at pH=7.4 and at different temperatures to show the size variation in response to temperature. In this method dilute synthesized microcarriers (1.0 mg/mL) were dispersed in PBS at pH=7.4.

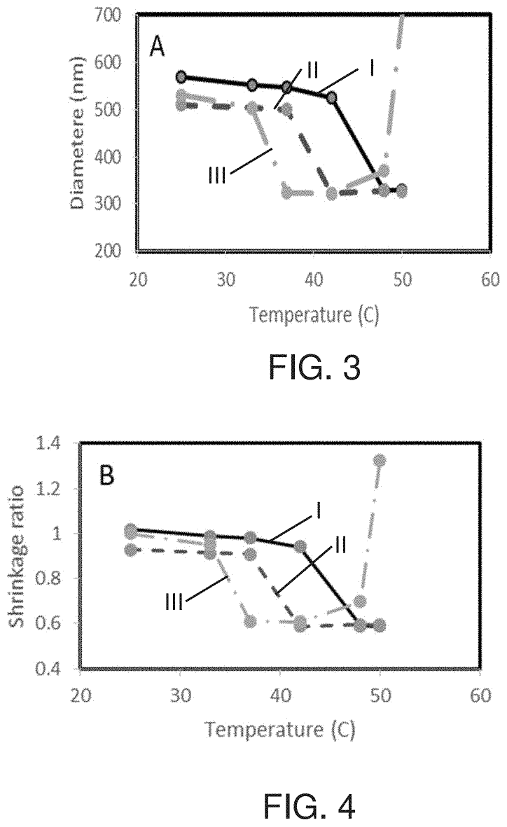

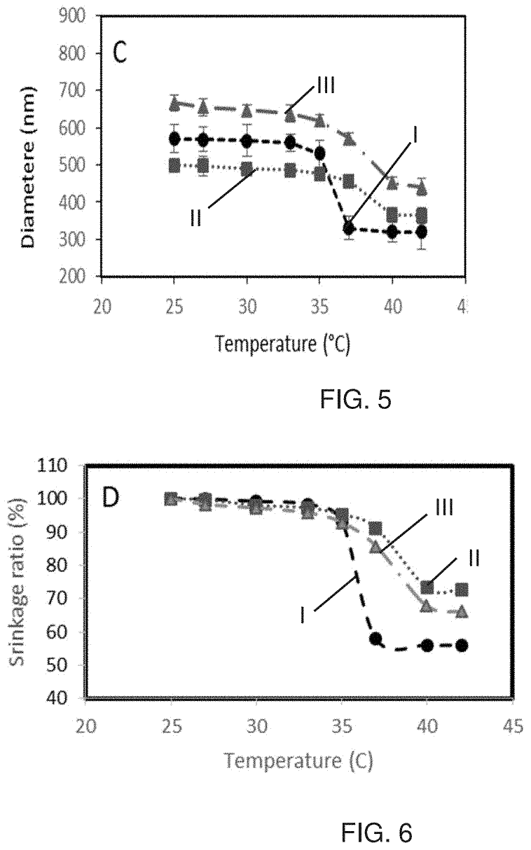

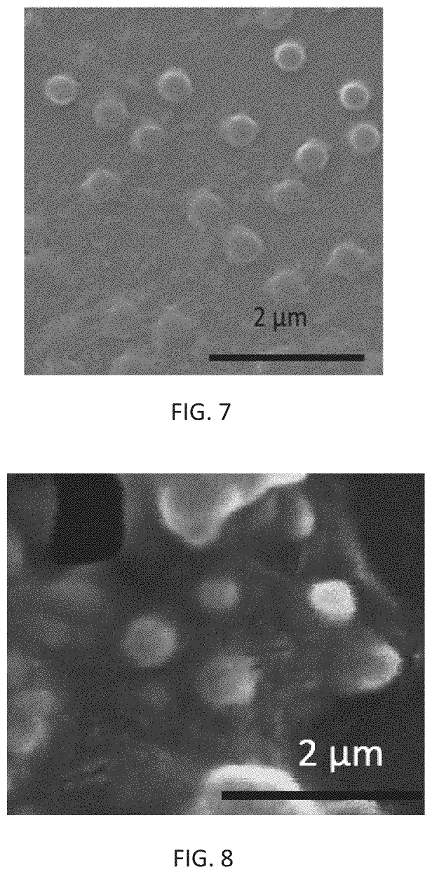

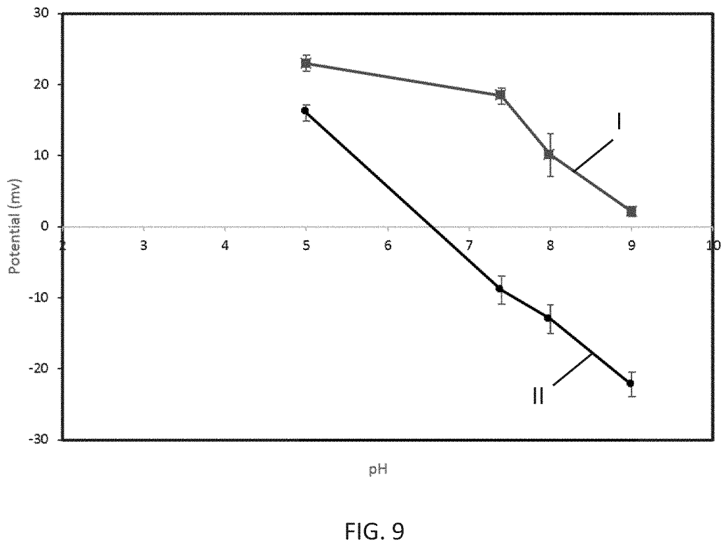

FIGS. 3 and 5 are graphic plots depicting hydrodynamic diameter variation of the stimulus-responsive carriers as a function of temperature, while FIGS. 4 and 6 are graphic plots depicting shrinkage ratio of the stimulus-responsive carriers as a function of temperature. As shown in FIGS. 3-6, hydrodynamic diameters of the microcarriers reduce with an increase in temperature from 25-42° C. Additionally, electrostatic repulsion due to presence of the carboxylic group of the acrylic acid (AA) causes peak shifts to larger hydrodynamic diameter (dh). This electrostatic repulsion is due to negative surface charge of the PNIPAM-AA microcarriers which is also obvious in Zeta potential profile of the microcarrier at pH 7.4 and pH=5, as depicted in FIG. 9. Presence of AA as a hydrophilic agent will lead to the LCST increment where LCST for PNIPAM and PNIPAM-AA is 32 and 34° C. respectively. Moreover, increasing the surface charge density in sample synthesized with SDS surfactant can result in delayed shrinkage, and hence LSCT increases to 36° C. This behavior is obvious in shrinkage ratio of these 3 microcarriers which has been depicted in FIGS. 4 and 6.

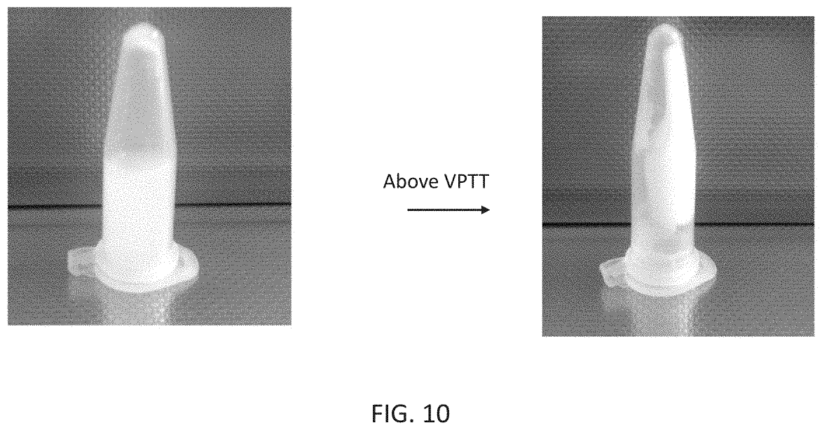

The shrinkage behavior of the stimulus-responsive carrier above its volume phase transition temperature (VPTT) tend to transform the carrier from sol phase to gel phase at relatively high concentrated colloidal microcarrier dispersions. This behavior is shown in FIG. 10 in which PNIPAM colloidal microcarrier is transformed to macro-gel above LSCT of PNIPAM-AA solution at pH=7. This behavior is so powerful property for different cell encapsulations in 3D cell culturing approaches.

Magnetic-pH sensitive nanogel is another element of the finally multi-responsive microparticles which can be used for various applications of smart drug delivery as well as 3D culturing and magnetic patterning of the mammalian cells. Here, magnetite containing chitosan nano/hybrid microcarrier was synthesized by electrostatic absorption of positive charged chitosan on magnetic nanoparticles. Resulting zeta potential of microcarrier depicted in FIG. 9 in which positive charge is obtained, presumably due to NH₃ groups of the chitosan and in the presence of H⁺ ion at acidic pH. These microcarrier is used for loading in the structure of the previous synthesized PNIPAM-AA microparticle with negative surface charge to provide multi-functional thermo-pH-magnetic responsive nano-hybrid stimulus-responsive carrier.

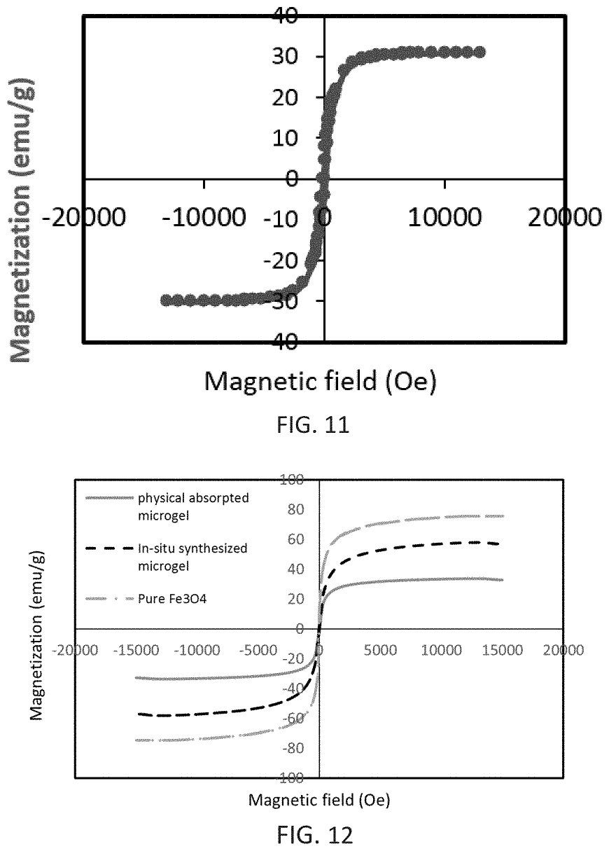

Magnetic characterizations of the free magnetite nanoparticle in comparison to magnetite-containing chitosan microcarrier is shown in FIGS. 11 and 12. It is obvious that magnetic saturation of the magnetite is decreased when is encapsulated in chitosan microcarrier. Moreover, VSM plot of magnetic-incorporated PNIPAM microcarriers prepared with both methods of physical absorption and in-situ synthesis are shown in FIG. 12, so that both of the microcarriers have less magnetization saturation compared to the pure nanoparticles.

Through using this multifunctional microcarrier different applications of desired cell and other bio-microparticles in the blood or other potential liquid biopsies is fulfilled. The pH-responsiveness of these microcarriers is visible in the presence of both acidic and basic environment so that in the case of acidic environment, Chitosan nanogel is protonated and hence the electrostatic repulsion of the same positive NH₄⁺ functional groups in the nanogel causes structural swelling and burst releasing of the whole content of the nanogel over the time. In the reverse case, i.e., in basic condition, microcarrier containing the pH responsive component can respond to pH increase by deprotonating the negative carboxylic groups, COO⁻ group so that the structural swelling is occurred in the same way.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A stimulus-responsive carrier, comprising:
a body of a polymeric component comprising a copolymer comprising units derived from a thermo-responsive monomer, a pH-responsive monomer, or any combination thereof, or a polymer comprising poly-N-vinylpyrrolidone, wherein the body of the polymeric component is thermo-responsive, pH-responsive, or thermo- and pH-responsive; and
a second component disposed within the body of the polymeric component, the second component comprising a hydrogel comprising a denatured protein, a synthetic hydrogel, or a combination thereof,
wherein the second component has a different composition than the polymeric component, and
wherein an average hydrodynamic diameter of the stimulus-responsive carrier decreases from 10-50% when the stimulus-responsive carrier undergoes a transition from a swollen, hydrated state to a shrunken, dehydrated state.

2. The stimulus-responsive carrier of claim 1, wherein the stimulus-responsive carrier has an average hydrodynamic diameter of 500 nm to 500 μm in the swollen, hydrated state.

3. The stimulus-responsive carrier of claim 1, wherein the copolymer comprises units derived from N-isopropylacrylamide, N-vinylpyrrolidone, 2-(2-methoxyethoxy)ethyl methacrylate, acrylic acid, allylamine, hydroxymethyl acrylamide, or any combination thereof.

4. The stimulus-responsive carrier of claim 1, wherein the hydrogel comprises methacrylated gelatin polymer (GeIMA), chitosan, collagen type I, collagen type IV, alginate, agarose, hyaluronic acid, elastin, poly(ethylene) glycol (PEG), poly(ethylene glycol) diacrylate (PEGDA), or any combination thereof.

5. The stimulus-responsive carrier of claim 1, wherein:
a single volume of the second component is disposed within the body of the polymeric component such that the second component is partially or entirely embedded within the body of the polymeric component, or
the second component is disposed within the body of the polymeric component as a plurality of discrete second component bodies, or
the second component is dispersed throughout the body of the polymeric component, thereby forming a mixture of second component molecules and polymeric component molecules.

6. The stimulus-responsive carrier of claim 1, further comprising one or more magnetic particles disposed within the body of the polymeric component or within the hydrogel of the second component.

7. The stimulus-responsive carrier of claim 6, wherein the one or more magnetic nanoparticles are coated with the hydrogel of the second component.

8. The stimulus-responsive carrier of claim 1, further comprising a targeting agent on an outer surface of the stimulus-responsive carrier, wherein the targeting agent is capable of binding to a target.

9. The stimulus-responsive carrier of claim 1, further comprising an active agent disposed within the body of the polymeric component or within the hydrogel of the second component.

10. The stimulus-responsive carrier of claim 9, wherein the active agent is a drug, a growth factor, a cytokine, an aptamer, a peptide, a dye molecule, or any combination thereof.

11. A biomedical implant, comprising the stimulus-responsive carrier of claim 9, wherein the stimulus-responsive carrier is temperature-responsive and a temperature increase releases the active agent from the stimulus-responsive carrier.

12. A method of using a stimulus-responsive carrier, the method comprising:
administering the stimulus-responsive carrier to a use environment, the stimulus-responsive carrier comprising
a body of a polymeric component comprising a copolymer comprising units derived from a thermo-responsive monomer, a pH-responsive monomer, or any combination thereof, or a polymer comprising poly-N-vinylpyrrolidone, wherein the body of the polymeric component is thermo-responsive, pH-responsive, or thermo- and pH-responsive, and
a second component disposed within the body of the polymeric component, the second component comprising a hydrogel comprising a denatured protein, a synthetic hydrogel, or a combination thereof, wherein the second component has a different composition than the polymeric component, and an average hydrodynamic diameter of the stimulus-responsive carrier decreases from 10-50% when the stimulus-responsive carrier undergoes a transition from a swollen, hydrated state to a shrunken, dehydrated state; and
applying a stimulus to the stimulus-responsive carrier, the stimulus comprising a temperature change, a pH change, or a combination thereof, thereby changing the average hydrodynamic diameter of the stimulus-responsive carrier.

13. The method of claim 12, wherein:
the stimulus-responsive carrier further comprises one or more magnetic nanoparticles disposed within the body of the polymeric component or within the hydrogel of the second component; and
applying the stimulus comprises applying a magnetic field, the magnetic field inducing a movement of the stimulus-responsive carrier.

14. The method of claim 12, where the use environment is a cell culture medium comprising cells, the method further comprising:
incubating the cell culture medium at an effective temperature for an effective period of time, whereby the cells proliferate and at least some of the cells adhere to the polymeric component, the hydrogel, or both the polymeric component and the hydrogel of the stimulus-responsive carrier; and
subsequently applying the stimulus, thereby changing the average hydrodynamic diameter of the stimulus-responsive carrier and releasing at some of the adhered cells from the stimulus-responsive carrier.

15. The method of claim 14, wherein the stimulus-responsive carrier further comprises an active agent disposed within the body of the polymeric component or within the hydrogel of the second component, the active agent comprising a drug, a cytokine, a growth factor, or a combination thereof.

16. The method of claim 14, wherein the stimulus-responsive carrier further comprises one or more magnetic nanoparticles disposed within the body of the polymeric component or within the hydrogel of the second component, and the method further comprises:
applying a magnetic field to induce movement of the stimulus-responsive carrier and the cells adhered thereto prior to subsequently applying the stimulus comprising a temperature change, a pH change, or a combination thereof.

17. The method of claim 16, further comprising isolating the stimulus-responsive carrier and the cells adhered thereto from the cell culture medium prior to subsequently applying the stimulus comprising a temperature change, a pH change, or a combination thereof.

18. The method of claim 12, wherein:
the use environment is a wound or a gastrointestinal tract;
the stimulus-responsive carrier further comprises an active agent; and
applying the stimulus releases at least a portion of the active agent into the wound.

19. A method of using a stimulus-responsive carrier, the method comprising:
administering the stimulus-responsive carrier to a biological sample comprising a target cell, the stimulus-responsive carrier comprising
a body of a polymeric component comprising a copolymer comprising units derived from a thermo-responsive monomer, a pH-responsive monomer, or any combination thereof, or a polymer comprising poly-N-vinylpyrrolidone, wherein the body of the polymeric component is thermo-responsive, pH-responsive, or thermo- and pH-responsive, and
a second component disposed within the body of the polymeric component, the second component comprising a hydrogel comprising a denatured protein, a synthetic hydrogel, or a combination thereof, wherein the second component has a different composition than the polymeric component,
one or more magnetic nanoparticles disposed within the body of the polymeric component or within the hydrogel of the second component, and
a targeting agent on an outer surface of the stimulus-responsive carrier, the targeting agent capable of binding to the target cell, wherein an average hydrodynamic diameter of the stimulus-responsive carrier decreases from 10-50% when the stimulus-responsive carrier undergoes a transition from a swollen, hydrated state to a shrunken, dehydrated state;
waiting an effective period of time allow binding of the targeting agent to the target cell, thereby forming a carrier-cell complex;
applying a magnetic field, thereby inducing movement of the carrier-cell complex; and
isolating the carrier-cell complex from the biological sample.

20. The method of claim 19, further comprising introducing the carrier-cell complex into a cell culture medium to induce proliferation of the target cell.
